(19) United States
(12) Patent Application Publication
(10) Pub. No.: US 2009/0131791 A1
(43) Pub. Date: May 21, 2009

(54) Ultrasonic Diagnostic Imaging System With Spectral and Audio Tissue Doppler

(75) Inventor: David W. Clark, Windham, NH (US)

Correspondence Address: PHILIPS INTELLECTUAL PROPERTY & STANDARDS, P.O. BOX 3001, Briarcliff Manor, NY, 10510-8001, US

(73) Assignee: KONINKLIJKE PHILIPS ELECTRONICS N.V., Eindhoven (NL)

(21) Appl. No.: 12/063,910

(22) PCT Filed: Aug. 18, 2006
(86) PCT No.: PCT/IB2006/052870

§ 371 Date: Feb. 15, 2008

Related U.S. Application Data
(60) Provisional application No. 60/710,429, filed on Aug. 22, 2005.
(60) Provisional application No. 60/749,214, filed on Dec. 8, 2005.

(51) Int. Cl. A61B 8/00

(52) U.S. Cl. 600/441

(57) ABSTRACT

A spectral tissue Doppler processor for an ultrasound system produces Doppler phase shift estimates of sequences of signal samples from a sample volume with a short-lag autocorrelator. The autocorrelation products are summed and an arc tangent taken of each sum to produce angle estimates. The angle estimates, which are proportional to the tissue motion velocity, are plotted, smoothed, and displayed as a spectral tissue Doppler display. The angle estimates are also used to produce the audio Doppler signal which is frequency-adjustable by a user. The spectral Doppler display exhibits good time and velocity resolution for motion which is less than that of blood flow such as myocardial motion. Major causes of blurring, unevenness, and distortion are reduced or eliminated.

This invention relates to medical ultrasonic diagnostic imaging systems and, in particular, to an ultrasound system which presents spectral and audio tissue Doppler information.

Tissue Doppler ultrasound is used in echocardiology to measure the motion and timing of the myocardium. It is an adaptation of the ultrasound techniques used for analyzing blood velocity: color flow mapping, and spectral and audio pulsed-wave Doppler. In the blood flow techniques, a clutter filter rejects the strong, slow tissue echo so that the very weak, faster echo from flowing blood can be seen. Tissue Doppler typically does not use a clutter filter because it is the strong, slow tissue echo that is of interest. In tissue Doppler it is the dominant slow tissue echo signal that is analyzed, which has an amplitude that is far above the signals from blood, noise, and reverberation.

Spectral Doppler signal processing for blood flow is based on overlapping short-time windowed Fast Fourier Transforms (FFTs), called a periodogram. The time resolution of a periodogram is directly proportional to the FFT time span, that is, the time over which a sequence of samples is acquired, and the velocity (Doppler frequency) resolution is inversely proportional to the FFT time span. This is a particularly difficult trade-off for tissue Doppler, because the slow velocity of tissue (compared to blood) requires good velocity resolution, while the dynamic timing of myocardial motion requires good time resolution. The resulting compromise spectrum is significantly blurred in both velocity and time when the periodogram is adapted for spectral tissue Doppler.

This spectral blurriness of tissue Doppler is not just an aesthetic issue. Clinicians are accustomed to measuring the peak velocity of a blood spectrum whose dominant sources of spread are blood turbulence, shear, and moving speckle. They also measure the peak velocity of the blurred tissue Doppler spectrum, not realizing that in this situation the spectral spread is entirely an artifact of the time and velocity resolution of the periodogram signal processing algorithm and the amplitude-to-gray-level mapping.

Like a two dimensional image, a Doppler spectrum has speckle from complex summation of echoes from many random scatterers. With the low velocity and time resolution demands of tissue Doppler, the bright speckle blobs and dark holes are large, often producing an uneven spectrum that further degrades aesthetics and measurements. The speckle can be partly alleviated by displaying a greater amplitude dynamic range, but that worsens the blurriness from the FFT time span.

Maintaining the Doppler sample gate in the myocardium for spectral tissue Doppler acquisition can be frustrating, because there is a lot of lateral motion during the heartbeat due to motion from the heart contractions and motion from patient breathing. However, the received signal is strong enough to be useable even from sidelobes when the myocardium is slightly outside the main beam because the echo reflections from tissue tend to be strong. But the motional effects can cause the spectral display to vary greatly in brightness and the audio to vary greatly in loudness. Automatic gain control can help this problem, but adds complexity.

When the signal strength fades due to the myocardium moving off the main Doppler beam, the stationary clutter from reverberation is more visible around the zero-velocity baseline, even though the myocardial signal is still dominant. A very low frequency clutter filter (for example, 25 Hz high pass) can make the display look cleaner, but this tends to make the desired signal discontinuous as it changes direction.

A problem with tissue Doppler audio is that the Doppler frequency is very low due to the relative low velocity of the myocardium and the low ultrasound frequency needed for adequate penetration. The very low pitch sounds are difficult for small loudspeakers to produce, and difficult for many human ears to hear. Turning up the volume helps somewhat, but can lead to distortion.

The conventional FFT periodogram technique is well suited to weak, broadband blood signals, but tissue Doppler always has one strong, narrowband signal. The technique typically used in color flow mapping to estimate an average velocity—taking the angle of a lag-1 autocorrelation estimate—is far better suited to narrowband analysis, and can be adapted to the continual stream of samples in tissue Doppler. The lag-1 autocorrelation is estimated over a moving time window of complex samples, producing a series of velocity (Doppler frequency) estimates that are far more precise in both velocity and time than can be achieved with FFT periodograms. The window may have a weighting function applied, such as a Hann (raised cosine) function.

In accordance with the principles of the present invention an apparatus and method are provided for spectral tissue Doppler processing to overcome time/frequency tradeoffs of the fast Fourier transform (FFT) by averaging a first or higher order lag of the autocorrelation and displaying a graphical plot as the spectral display in pulsed Doppler form. The phase shifts estimated in this manner are used to synthesize an audio output which is scaleable to a higher frequency. The resultant spectral and audio tissue Doppler ultrasound is greatly improved in resolution, accuracy, uniformity, and clarity by the use of narrowband autocorrelation processing instead of conventional broadband FFT periodogram processing.

In the Drawings:

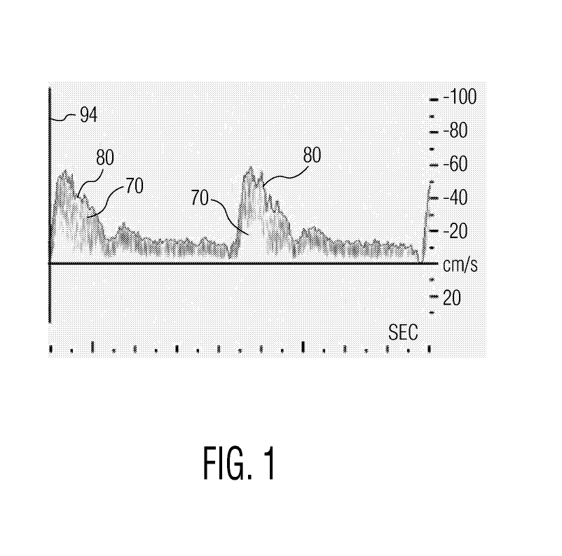

FIG. 1 illustrates a conventional spectral display of blood flow.

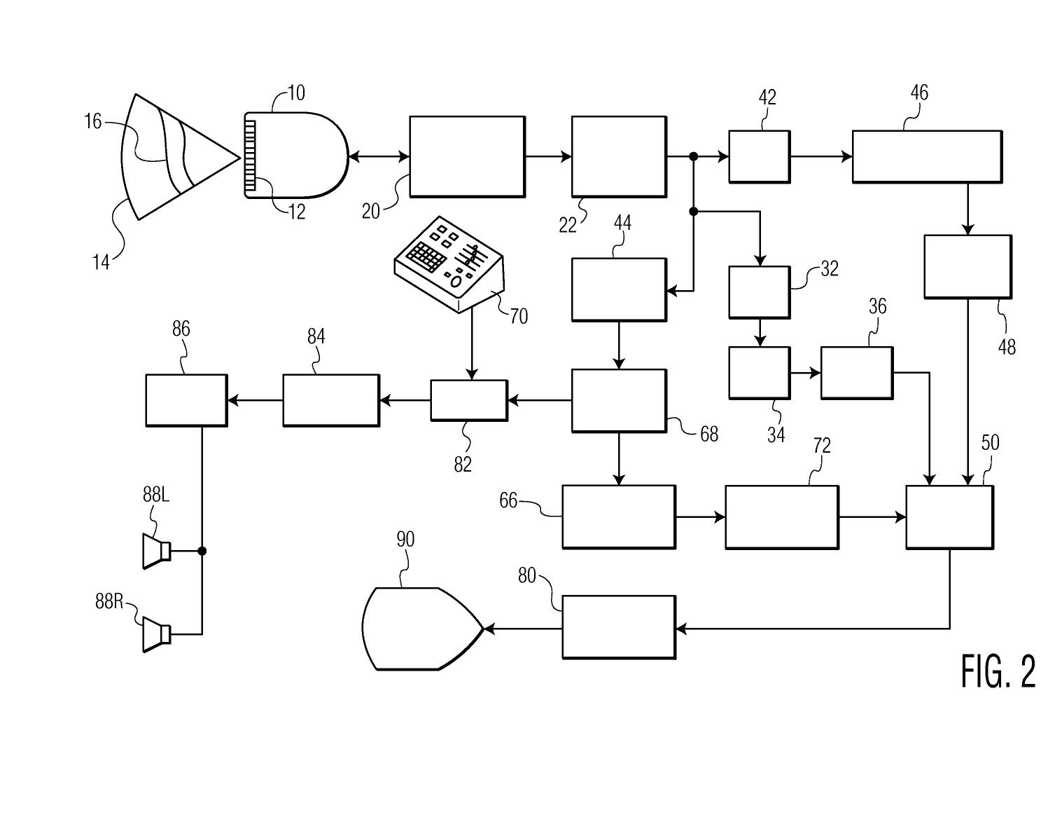

FIG. 2 illustrates in block diagram form an ultrasonic diagnostic imaging system constructed in accordance with the principles of the present invention.

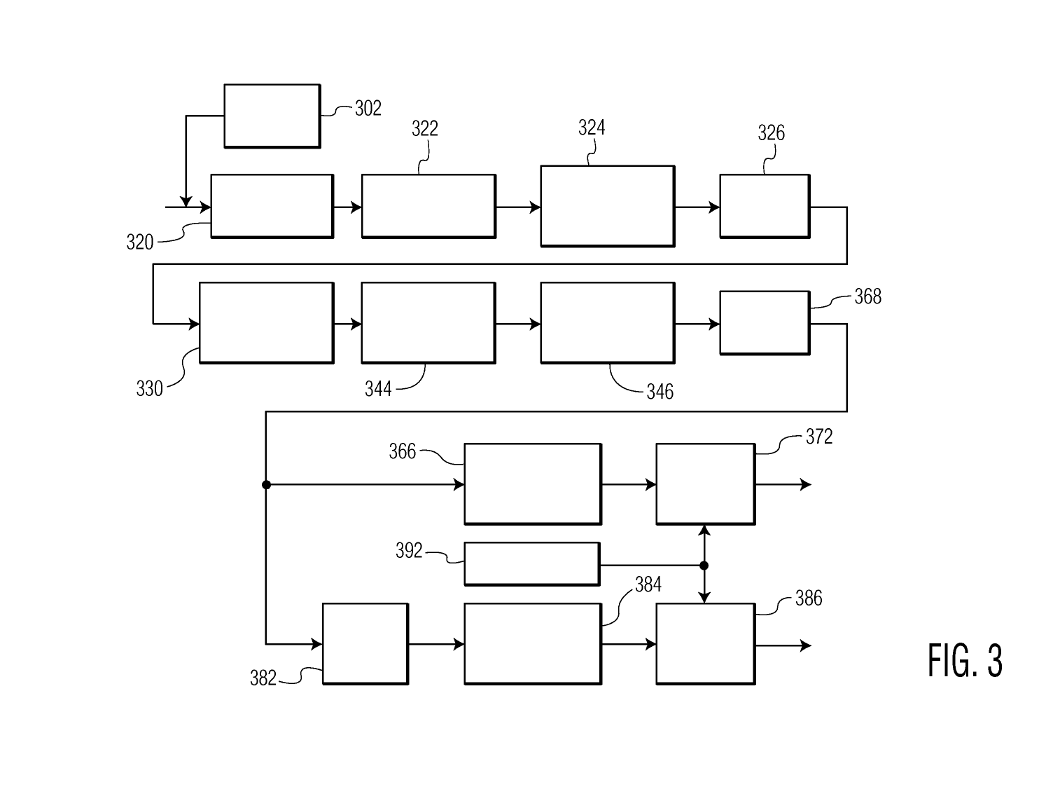

FIG. 3 is a functional block diagram of a spectral and audio tissue Doppler processor constructed in accordance with the principles of the present invention.

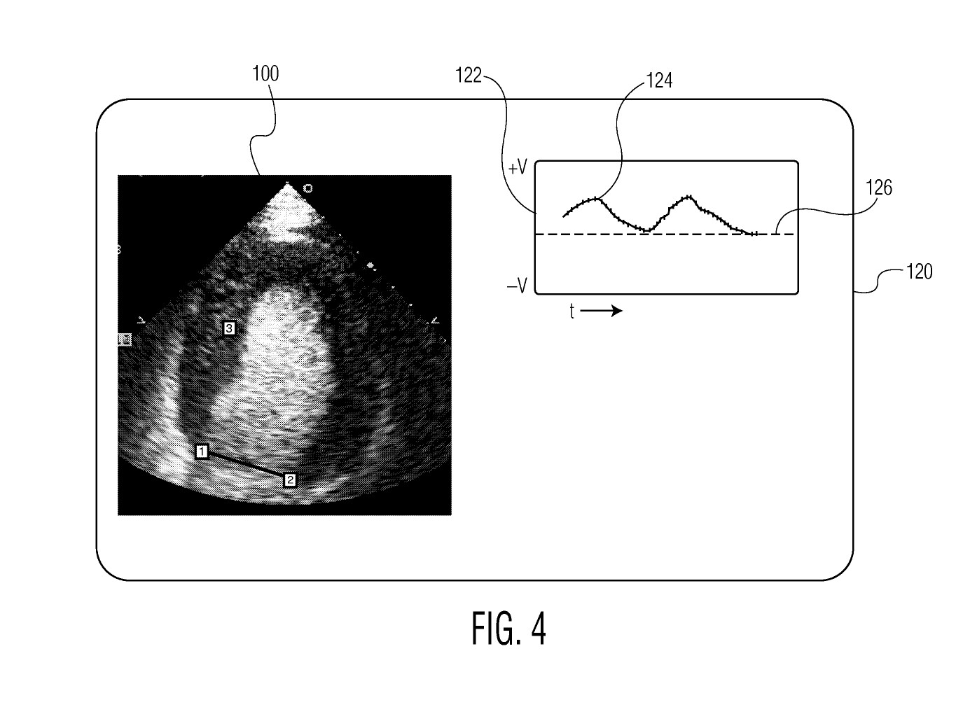

FIG. 4 illustrates a display screen of an ultrasound system when performing spectral tissue Doppler analysis of the myocardium.

Referring first to FIG. 1 a standard spectral Doppler display of blood flow is shown. The scale of the vertical axis 94 of the spectral display is in cm/sec and the scale of the horizontal axis is time (sec.) The spectral display is produced by acquiring a sequence of samples from a point (sample volume) in a chamber of the heart or blood vessel. A group of successive samples, referred to as a window, is operated on as a unit. For instance a window for blood flow spectral Doppler may consist of 128-256 consecutive samples. The samples of the window are generally weighted with the greatest weighting used in the center of the window. The weighted samples then undergo FFT processing which produces a Fourier series of weighted samples as is known in the art. FFT processing transforms the time domain samples into frequency domain samples which are complex samples having both real and imaginary parts. The magnitudes of the samples are computed and a logarithmic value is taken of each magnitude value. Each log value is mapped to a grayscale level for a vertical column of the spectral display. In this way the frequency domain samples of a window are displayed as a column of data which forms one line of the scrolling spectral display. As soon as one line of the spectral display is formed another window of samples is acquired and FFT processed in the same way. Usually there is overlap between consecutive windows to limit the variation between successive FFT results, so that the temporal variations are adequately sampled without aliasing. As FIG. 1 shows, the spectral lines 70 are computed rapidly and follow one another closely as to appear as a continuum of gray. For blood flow the clinician is often interested in the peak velocity of the blood flow as may occur with a jet from a leaking heart valve. These peak values may be acquired by tracing the peaks of the spectral lines, either manually or automatically as described in U.S. Pat. No. 5,287,753 (Routh et al.), as illustrated by the peak trace 80 in FIG. 1.

When the FFT processor is used for spectral tissue Doppler rather than blood flow, the lower speed of the tissue motion must be taken into consideration. This dictates that the rate of sample acquisition be reduced (lower pulse repetition frequency, PRF). Hence the sample window for spectral tissue Doppler is usually shorter, such as 64 samples instead of the 128-256 samples used for blood flow. The shorter window length results in reduced velocity resolution, whereas the desire for tissue Doppler is better resolution because the range of velocities encountered is much less for tissue than for blood flow. The reduced PRF also means that the time interval from window to window is generally greater due to the length of time to acquire the necessary samples at the lower acquisition rate. Thus, time resolution is reduced, impairing the ability to detect subtle timing differences of different regions of tissue such as the lateral wall and septum of a heart chamber. The blurring resulting from these processing factors may not be apparent to the clinician who is accustomed to seeing similar appearances in spectral Doppler blood flow spectrograms, which are caused by physiological rather than processing effects.

An ultrasound system constructed in accordance with the principles of the present invention is shown in block diagram form in FIG. 2. A transducer probe 10 transmits ultrasonic beams and receives echoes in response from an image region 14 of a subject which contains organs and blood vessels 16. In this particular example the ultrasound system is to be used to analyze the motion of tissue in the body such as the movement of the myocardium of the heart. The ultrasonic energy is transmitting and received by elements of a transducer array 12. The received echoes are coupled to a beamformer 20 which produces coherent echo signals. These echo signals are demodulated by a quadrature demodulator 22. In the system shown in this example the echo signals are processed further in three different ways. Amplitude detector 32 performs an amplitude detection of the received echo signals and the detected signals are compressed by a log compression 34. The echo values are then mapped to display values by a greyscale map 36. This processing will produce a B mode image of the moving tissue of interest, in this example, the myocardium. The quadrature demodulated signals are filtered by a wall filter 42 to remove signals from stationary tissue, and ensembles of the filtered signals undergo Doppler processing by a color Doppler processor 46. The Doppler processor 46 can produce different selectable representations of motion such as velocity, acceleration, variance or Doppler power. The resultant phase shift or intensity estimates are mapped to corresponding colors or hues by a color map 48. This processing produces a two or three dimensional color overlay of the tissue motion which can be aligned with and overlay the structural B mode image. As mentioned above, for a tissue Doppler signal where the signals of interest are strong and of low velocity, a wall filter may not always be necessary. The B mode image and the color motion image are coupled to a scan converter 50 where they are combined in the desired spatial format for display as a two or three dimensional color tissue Doppler image.

In accordance with the principles of the present invention the quadrature demodulated signals from a selected sample volume are coupled to an autocorrelator 44. The autocorrelator may be of an adjustable lag order. In this example the autocorrelator is set to be a lag-1 autocorrelator and operates to multiply an echo sample from the sample volume by the complex conjugate of the previous sample, an operation which may be expressed as Sₙ₊₁*conj [Sₙ]. The autocorrelator operates on the echo samples in windowed groups. For instance, a sixty-four sample window can be used. The samples in the window are generally weighted with the higher weighting functions used in the center of the window. The window will generally overlap for the desired time resolution. For instance the first window may include samples 1-64, the second window samples 16-80, the third window samples 32-96, and so on. When a higher PRF is employed lag-2 autocorrelation may be preferable, which would operate on every other sample in the sequence. The lag-1 multiplication will yield a relatively imprecise phase shift angle estimate at a very precise time, the time interval of the two consecutive samples. The autocorrelator 44 increases the angle estimate precision by summing the products of the window and taking the angle of the result, which is expressed as a complex number having a real and an imaginary part. This angle estimate is applied to arc tan. calculator 68 which looks up or calculates the phase shift angle value to be used for the tissue Doppler spectral display, as the tissue velocity is proportional to the phase shift angle determined by the arc tangent of the autocorrelation result.

In this manner a sequence of windowed autocorrelation velocity estimates are produced sequentially in time. These velocity estimates can be plotted in the manner of a curve of a graph which conceptually is how the spectral display is formed. The velocity estimates are applied to an interpolator 66 which forms a smooth curve of the sequence of data points. The resultant curve is coupled to a graphics processor which puts the curve on a familiar spectral display plot. The spectral display will thus be in the format familiar to clinicians. In this example the graphical spectral tissue Doppler image is coupled to the scan converter 50 for display alongside the color tissue Doppler image as shown in FIG. 4 below. The images produced by the scan converter are coupled to a video processor 80 for display on an image display 90.

In accordance with another aspect of the present invention the velocity estimates produced by the arc tan. calculator 68 are coupled to a controllable scaler 82. The scaler 82 is responsive to a control signal from a user control panel 70 to set a scaling factor which scales the applied velocity estimates. Since these values represent an angle produced by the average phase shift from one sample to another, they comprise phase shift values proportional to the Doppler frequency. These phase shift values are multiplied by the selected scaling factor to obtain the audio output frequency, which is a phase shift from one audio output sample to the next. The scaled values are smoothed by an interpolator 84, then applied to an audio processor 86, which generates sinusoidal audio signal samples, where the phase shift between audio samples is the interpolated, scaled phase shift. The audio processor drives stereo speakers 88L and 88R on the ultrasound system with signals dependent on the sign of the phase shift (frequency): positive frequencies, corresponding to motion toward the transducer, are provided to one speaker and negative frequencies, corresponding to motion away from the transducer, are provided to the other. A stereo audio Doppler signal is thereby produced. The clinician will listen to the audio Doppler signal for changes in the pitch of the tone. Since the absolute value of the tone is not important but only changes in the tone, the clinician can adjust the user control to adjust the audio Doppler scaling value to produce a range of pitches best suited to his or her ear. Furthermore, since the audio Doppler sinusoid is produced from a frequency determination and not a signal amplitude, there is no fading or dropout as is the case with FFT-provided audio Doppler.

Referring to FIG. 3 a functional block diagram of an example of the present invention is shown. The transducer elements of a transducer probe (10, FIG. 1) is driven by a transmit pulser (302) for ultrasound pulse transmission and the echoes received by the transducer are coupled to a receive beamformer (320). The beamformed echo signals are demodulated to a baseband by a demodulator (322) and echo samples from the sample volume where the spectral tissue Doppler measurement is being made are summed by a sample gate accumulator (324), then stored for processing in a memory buffer (326). That is, what is stored for further processing is one complex number for each transmit event, where the complex number represents the sum of demodulated echo signal over the sample volume range. The stored samples are selected for processing in overlapping time windows (330) which undergo short-lag autocorrelation (344). The autocorrelation estimate is inherently power-weighted, so samples with speckle cancellation contribute very little variation compared to samples that are stronger. Furthermore, by using only the angle of the autocorrelation estimate, there is no amplitude fading, blooming or speckle in the result. This makes the inventive technique much more reliable without undue adjustment, with less need for the user to adjust the system controls.

There is no need for a clutter filter with this technique. When the myocardium is slightly off the main beam, it is still by far the dominant signal compared to the clutter, so the spectral display is negligibly affected. When there is no tissue signal present, the correlation angle from noise will be random. This would create a distracting spectral and audio result, so the angle needs to be set to zero when a tissue signal is not detected. One approach is to compare the power or correlation magnitude to a threshold (346). Another approach is to compare the coherence (|R1|/R0, where R1 is the lag-1 auto-correlation, and R0 is the lag-0 autocorrelation or variance) to a threshold, since the tissue signal is very coherent, while the noise is very incoherent. The arc tangent is taken of the validated autocorrelation values (368).

The spectral display is produced by interpolating the precise velocity estimates from the autocorrelation angles (366) to make a connected curve. The spectrum is actually more of a graph than a conventional spectrum, but it conveys the essential information without the conventional artifacts. In fact, it will look familiar to clinicians who analyze a stored loop of tissue color images to produce similar velocity vs. time graphs. The smoothness and thickness of the spectral graph (372) is controllable by the span of the autocorrelation estimation time window. The video intensity of the graph can be adjusted with a gain control (392), without any signal-dependent variation.

The audio output is a synthesized sinusoidal signal having an instantaneous frequency that is smoothly interpolated (384) between the Doppler frequency estimates from the autocorrelation angles. The sinusoid (386) is directed to one of the stereo channels based on the sign of the frequency. The stereo is blended for sub-sonic frequencies to avoid an audio discontinuity when the velocity changes direction. The amplitude (loudness) of the sinusoid can be adjusted with the gain control (392), without any signal-dependent variation.

Since the audio is simply a sinusoid produced from the series of frequency numbers, the frequency (pitch) can be easily scaled by an arbitrary factor. This greatly alleviates the problem of low-pitch insensitivity of speakers and ears. Clinicians do not depend on the absolute pitch of the audio, but rather the variations in pitch. The pitch scaling (382) that this technique offers is limited only by what sounds pleasing. It is easily set by a user-controlled adjustment.

A further refinement of this technique is to increase the Doppler pulse repetition frequency (PRF) by some integer factor and increase the autocorrelation lag (in samples) by the same factor. The transmit power will probably decrease by the same factor, but for tissue Doppler the signal to noise ratio is very high. The increased averaging from having more samples in each autocorrelation estimate can outweigh the decreased transmit power.

A typical screen display 120 produced by the ultrasound system of FIG. 2 is shown in FIG. 4. The combined B mode and color Doppler image 100 in this example is a tissue Doppler image of the left ventricle, with the mitral valve plane indicated by the line between points 1 and 2 at the bottom of the image. A sample volume 3 is positioned on the myocardium of the septal wall. The spectral tissue Doppler display 122 taken at the sample volume 3 is shown to the right of the color tissue Doppler image 100. The spectral line display 124 is shown over the zero velocity baseline 126 of the spectral display 122.

What is claimed is:

1. An ultrasonic diagnostic imaging system which produces a spectral display of tissue motion at a sample volume comprising:
a transducer probe;
a lag-n autocorrelator responsive to echo samples emanating from the sample volume which operates on windows of echo samples from temporally different transmits to produce autocorrelation values;
an arc tangent processor, responsive to the autocorrelation values, which produces angle estimate values;
a graphics processor, responsive to the angle estimate values, which produces a spectral tissue Doppler image; and
a display, coupled to the graphics processor, which displays the spectral tissue Doppler image.

2. The ultrasonic diagnostic imaging system of claim 1, further comprising an interpolator, responsive to the angle estimate values and coupled to the graphics processor, which produces an angle estimate curve.

3. The ultrasonic diagnostic imaging system of claim 1, further comprising a threshold processor, responsive to the autocorrelation values, which validates autocorrelation values against a threshold.

4. The ultrasonic diagnostic imaging system of claim 1, further comprising:
an audio processor, responsive to angle estimate values, which produces an audio sinusoid at an output; and
a loudspeaker, coupled to the audio processor output, which produces an audio Doppler output.

5. The ultrasonic diagnostic imaging system of claim 4, further comprising:
a scaler, coupled to receive angle estimate values and responsive to a user control and having an output coupled to the audio processor, which produces scaled angle estimate values.

6. The ultrasonic diagnostic imaging system of claim 5, further comprising:
an interpolator having an input coupled to receive scaled angle estimate values and an output coupled to the audio processor.

7. The ultrasonic diagnostic imaging system of claim 4, wherein the loudspeaker comprises stereo loudspeakers responsive to the audio processor output in correspondence to the sign of the frequency of the audio sinusoid.

8. The ultrasonic diagnostic imaging system of claim 1, further comprising:
a B mode processor responsive to echo samples emanating from a region of tissue which acts to produce a B mode image;
a color Doppler processor responsive to echo samples emanating from the region of tissue which acts to produce a color image of tissue motion; and
a scan converter, responsive to the B mode image and the color image of tissue motion, and having an output coupled to the display, which acts to produce a color tissue Doppler image.

9. The ultrasonic diagnostic imaging system of claim 8, wherein the display is operable to simultaneously display the spectral tissue Doppler image and the color tissue Doppler image.

10. The ultrasonic diagnostic imaging system of claim 9, further comprising an audio processor, responsive to the autocorrelation values, which acts to produce an audio Doppler signal corresponding to the spectral tissue Doppler image.

11. The ultrasonic diagnostic imaging system of claim 1, wherein the lag-n autocorrelator comprises a lag-1 autocorrelator.

12. The ultrasonic diagnostic imaging system of claim 1, wherein the lag-n autocorrelator comprises a lag-2 autocorrelator.

13. A method of producing a spectral tissue Doppler display from a sequence of echo signals received from a sample volume located on moving tissue comprising:
demodulating the echo signals to preserve their phase information;
forming overlapping time windows of sequences of demodulated echo signals from temporally different transmits;
performing a short-lag autocorrelation of the windows of echo signals;
producing an arc tangent angle estimate for each autocorrelated window; and
producing a spectral display from the arc tangent angle estimates.

14. The method of claim 13, further comprising:
interpolating the angle estimates; and
wherein producing a spectral display comprises producing a spectral display from the interpolation of the angle estimates.

15. The method of claim 14, further comprising:
comparing results of the short-lag autocorrelation against a power or coherence threshold.

16. The method of claim 14, further comprising:
coupling the angle estimates to an audio processor which produces an audio sinusoid; and
producing an audio Doppler signal from the audio sinusoid.

17. The method of claim 16, further comprising adjusting the audio Doppler signal by adjustment of the frequency of the sinusoid.

18. The ultrasonic diagnostic imaging system of claim 3, wherein the threshold comprises a power threshold.

19. The ultrasonic diagnostic imaging system of claim 3, wherein the threshold comprises a coherence threshold.
